(19) United States
(12) Patent Application Publication
(10) Pub. No.: US 2022/0180965 A1
(43) Pub. Date: Jun. 9, 2022

(54) ANALYSIS METHOD AND ANALYSIS PROCESSING APPARATUS FOR LOSS OF HETEROZYGOSITY (LOH) OF HUMAN LEUKOCYTE ANTIGEN (HLA)

(71) Applicants: SHENZHEN TISSUEBANK PRECISION MEDICINE CO., LTD, Shenzhen (CN); SHANGHAI TISSUEBANK MEDICAL LABORATORY CO., LTD, Shanghai (CN); SHANGHAI TISSUEBANK BIOTECHNOLOGY CO., LTD, Shanghai (CN); SHANGHAI TISSUEBANK GENE TECHNOLOGY CO., LTD, Shanghai (CN)

(72) Inventors: Zhongzheng Zheng, Shenzhen (CN); Qingqing He, Shenzhen (CN); Kuanzhen Liao, Shenzhen (CN); Xiaonian Tu, Shenzhen (CN); Zhiyang Yuan, Shenzhen (CN)

(21) Appl. No.: 17/522,841

(22) Filed: Nov. 9, 2021

Related U.S. Application Data
(63) Continuation of application No. PCT/CN2021/085090, filed on Apr. 1, 2021.

(30) Foreign Application Priority Data
Dec. 4, 2020 (CN) 202011404354.4

(51) Int. Cl. G16B 20/20; G16B 20/00; G16B 30/10; G16B 30/20

(52) U.S. Cl. CPC G16B 20/20; G16B 30/20; G16B 30/10; G16B 20/00

(57) ABSTRACT

The present application belongs to the field of bioinformatic analysis, and discloses an analysis method and analysis processing apparatus for loss of heterozygosity (LOH) of human leukocyte antigen (HLA). Directed to the detection demands for relapse after transplantation caused by HLA Loss and based on Next Generation Sequencing (NGS) data, the present application provides an analysis method and analysis processing apparatus for HLA Loss, which is capable of conveniently achieving the flow and in-batch operation. The present application has low workload of artificial interpretation, and can accurately detect the presence of HLA Loss or not in a sample and thus has significant meaning to the relapse after transplantation caused by HLA Loss.

### CROSS-REFERENCE TO RELATED APPLICATION

The present application claims the priority of a Chinese patent application 202011404354.4, filed on Dec. 4, 2020, the entirety of which is incorporated herein by reference.

### TECHNICAL FIELD

The present application relates to the field of bioinformatic analysis, and in particular to an analysis method and analysis processing apparatus for loss of heterozygosity (LOH) of human leukocyte antigen (HLA).

### BACKGROUND

Loss Of Heterozygosity (LOH) is a common chromosomal aberration in malignant tumors. LOH occurring in human leucocyte antigen (HLA) chromosome region is called HLA Loss; the occurrence of HLA Loss will influence the normal expression of HLA, such that tumor cells escape the killing from immune cells of a body, thus leading to an immune escape of tumor cells. HLA Loss often occurs in solid tumors, e.g., non-small cell lung cancer, and also occurs in hematologic tumors. If tumor cells suffer from HLA Loss after the transplantation of hematopoietic stem cells, the tumor cells escape the killing from immune cells, leading to the relapse of such kind of hematological disease. Up to 2017, in haploidentical hematopoietic stem cell transplantation cases, the relapse after transplantation caused by HLA Loss has occupied 33% of all the relapse cases. The results of HLA Loss International Multicencer Cooperative Team show that in 396 valid cases, there are 51 cases of HLA Loss-caused relapse, of which 35 cases are from haploidentical transplantation, 12 cases are from HLA-mismatched unrelated donor transplantation, and 4 cases are from identical unrelated donor transplantation, and there is no HLA Loss of cord blood transplantation. The research results of the Multicencer show that HLA Loss is one of the major mechanisms to cause immune escape and relapse after hematopoietic stem cell transplantation, and the ratio of HLA Loss is associated with the quantity of the mismatched sites of HLA between a donor and a patient.

After transplantation, the detection of HLA Loss has important significance to make clear the relapse reason and direct the relapse treatment. Due to the missing of mismatched sites, conventional relapse treatment means, such as, to reduce the dosage of immunosuppressors and donor lymphocyte infusion (DLI), are not applicable and should be strictly prohibited in HLA Loss relapse. Because DLI infusion will not produce graft-versus-leukemia effect, but will produce severe graft-versus-host disease effect in HLA Loss relapse in the relapse of HLA Loss. Based on this, the presence of HLA Loss after relapse after transplantation or not should become a conventional clinical project; and even, HLA Loss dynamic monitoring should be performed before the relapse to predict the relapse as early as possible, which is also crucial to the formulation of a therapeutic regimen for the relapse.

HLA Loss-caused relapse after transplantation has been a newly occurred phenomenon in recent years. At present, there is few of transplantation center for the detection of HLA Loss on a international scale; partial institutions attempt using a fluorescent quantitative PCR method for detection, but the method can only cover the HLA types of about 70% population. There is even no study on the HLA Loss-caused relapse after transplantation at home, and there is no efficient, accurate, mature and reliable detection method and system for detection and analysis. Based on the above reasons, it is urgent to provide an efficient, accurate, mature and reliable HLA Loss detection method and system suitable for all the people at present.

### SUMMARY

### Technical Problem

Directed to the demands for the detection of HLA Loss-caused relapse after transplantation, the present application provides an efficient, accurate, mature and reliable HLA Loss detection method and analysis processing apparatus suitable for all the people at present.

### Technical Solutions

For the purpose, one aspect of the present application is to provide an analysis method for HLA Loss, including the following steps:

1, splitting and filtering a sample data to obtain a filtered sample sequence;
2, acquiring a sequencing data file of each HLA gene, wherein the sequencing data file comprises the sample data of each HLA gene;
3, generating a first allele sequence as a reference gene before recipient transplantation;
4, acquiring a percentage occupied in each HLA gene after the recipient transplantation;
5, acquiring a percentage of the HLA gene chromosome region after the recipient transplantation, denoted by HLA %;
6, acquiring a total percentage of each chromosome in a cell after the recipient transplantation, denoted by STR %;
7, judging a negative or positive result of HLA Loss after the recipient transplantation.

In embodiments of the present application, each step is specifically as follows:

1, splitting and filtering a sample downlink data after sequencing to obtain the filtered sample sequence;
2, based on a given alignment parameter, aligning the filtered sample sequence to the reference gene sequence of each HLA gene, and splitting the aligned sequencing sequence to each HLA sequencing data file; the sequencing data file comprises the sample downlink data of each HLA gene after sequencing;
3, acquiring the type of each HLA allele from a recipient and a donor before transplantation, after alignment, obtaining a single nucleotide polymorphism (SNP) difference of each HLA gene between the recipient and the donor, and generating the first allele sequence of the HLA genotype before recipient transplantation as the reference gene;
4, aligning the sequencing data file to the reference gene, performing statistics on respective sequencing depths of the recipient and the donor in a position SNP of each HLA gene, and averaging a depth frequency of the recipient in all the SNP positions of each HLA gene to obtain the percentage occupied in each HLA gene after the recipient transplantation;
5, averaging the percentage of the recipient in each HLA gene, namely, a percentage of HLA chromosome region after the recipient transplantation, denoted by HLA %;
6, acquiring a total percentage of each chromosome in a cell after the recipient transplantation, denoted by STR %.
7, negative or positive judgment: when HLA %≤0.5% and STR %≥3%, HLA Loss is judged positive; when HLA %≥3%, HLA Loss is judged negative; when 0.5%≤HLA %<3%, judgment may not be performed, and the cell HLA chromosome may be in a deficiency phase after the recipient transplantation.

In one embodiment of the present application, the analysis method includes the following steps:

8, outputting the percentage and average percentage of each HLA gene, negative or positive judgment result to a report file.

In one embodiment of the present application, each HLA gene is respectively HLA-A, HLA-B, HLA-C, HLA-DRB1, HLA-DQB1 and HLA-DPB1.

In one embodiment of the present application, the HLA-A gene is aligned to A*01:01:01:01; the HLA-A gene is aligned to B*07:02:01:01; the HLA-C gene is aligned to C*01:02:01:01; the HLA-DRB1 gene is aligned to DRB1*01:02:01:01; the HLA-DQB1 gene is aligned to DQB1*05:01:01:01; and the HLA-DPB1 gene is aligned to DPB1*01:01:01:01.

Another aspect of the present application is to provide an analysis processing apparatus for HLA Loss, including the following modules:

1, a splitting and filtering module, used for splitting and filtering a sample data to obtain a filtered sample sequence;
2, a gene sequencing data file acquisition module, used for acquiring each HLA gene sequencing data file, wherein the sequencing data file comprises the sample data of each HLA gene;
3, a reference gene generation module, used for generating a first allele sequence of HLA genotype as a reference gene before recipient transplantation;
4, a calculation module for a percentage in each HLA gene, used for acquiring a percentage occupied in each HLA gene after the recipient transplantation;
5, a calculation module for a percentage of a HLA gene chromosome region, used for acquiring a percentage of a HLA gene chromosome region after the recipient transplantation, denoted by HLA %;
6, a calculation module for a total percentage of each chromosome of each HLA gene, used for acquiring a total percentage of each chromosome in a cell after the recipient transplantation, denoted by STR %;
7, a negative or positive judgment module, used for judging a negative or positive result of HLA Loss after the recipient transplantation.

In one embodiment of the present application, each module is specifically as follows:

1, a splitting and filtering module, used for splitting and filtering a sample downlink data after sequencing to obtain the filtered sample sequence;
2, a gene sequencing data file acquisition module, used for aligning the filtered sample sequence to the reference gene sequence of each HLA gene based on a given alignment parameter, and splitting the aligned sequencing sequence to each HLA sequencing data file; wherein the sequencing data file comprises the sample downlink data of each HLA gene after sequencing;
3, a reference gene generation module, used for acquiring a type of each HLA allele from a recipient and a donor before transplantation, after alignment, obtaining an SNP difference of each HLA gene between the recipient and the donor, and generating a first allele sequence of the HLA genotype as the reference gene before recipient transplantation;
4, a calculation module for a percentage of each HLA gene, used for aligning the sequencing data file to the reference gene, performing statistics on respective sequencing depths of the recipient and the donor in a position SNP of each HLA gene, and averaging a depth frequency of the recipient in all the SNP positions of each HLA gene to obtain the percentage occupied in each HLA gene after the recipient transplantation;
5, a calculation module for a percentage of the HLA gene chromosome region, used for averaging the percentage of the recipient in each HLA gene, namely, a percentage of the HLA chromosome region after the recipient transplantation, denoted by HLA %;
6, a calculation module for a total percentage of each chromosome of each HLA gene, used for acquiring a total percentage of each chromosome in a cell after the recipient transplantation, denoted by STR %;
7, a negative or positive judgment module: used for judging a negative or positive result of HLA Loss after recipient transplantation; when HLA %≤0.5% and STR %≥3%, HLA Loss is judged positive; when HLA %≥3%, HLA Loss is judged negative; when 0.5≤% HLA %<3%, judgment may not be performed, and the cell HLA chromosome may be in a deficiency phase after the recipient transplantation.

In one embodiment of the present application, the analysis processing apparatus further includes the following modules:

8, a report file generation module, used for outputting the percentage and average percentage of each HLA gene, and negative or positive judgment result to a report file.

In one embodiment of the present application, each HLA gene is respectively HLA-A, HLA-B, HLA-C, HLA-DRB1, HLA-DQB1 and HLA-DPB1.

In one embodiment of the present application, the HLA-A gene is aligned to A*01:01:01:01; the HLA-A gene is aligned to B*07:02:01:01; the HLA-C gene is aligned to C*01:02:01:01; the HLA-DRB1 gene is aligned to DRB1*01:02:01:01; the HLA-DQB1 gene is aligned to DQB1*05:01:01:01; and the HLA-DPB1 gene is aligned to DPB1*01:01:01:01.

### Beneficial Effects

Based on the above description, the analysis method and analysis processing apparatus provided by the present application based on Next Generation Sequencing (NGS) data can conveniently achieves the flow and in-batch operation. The present application has low workload of artificial interpretation, and can accurately detect the presence of HLA Loss or not in a sample and thus has significant meaning to the relapse after transplantation caused by HLA Loss.

### BRIEF DESCRIPTION OF THE DRAWINGS

FIG. 1 shows a flow diagram of an analysis method of the present application.

FIG. 2 is a display diagram showing a mutual relation of each module in an analysis processing apparatus of the present application.

FIG. 3 is a display diagram showing integrated data quality of original data in Example 1.

FIG. 4 is a display diagram showing integrated data quality of filtered original data in Example 1.

FIG. 5 is a display diagram showing integrated data quality of original data in Example 2.

FIG. 6 is a display diagram showing integrated data quality of filtered original data in Example 2.

FIG. 7 is a diagram showing a linear fitting curve in Example 3.

### DETAILED DESCRIPTION OF THE EMBODIMENTS

The present application will be further described by the following examples, which is construed as specifying the present application instead of limiting the present application. It should be indicated that a person skilled in the art may make several improvements and modifications within the principle of the present application. These improvements and modifications should fall within the protection scope of the present application.

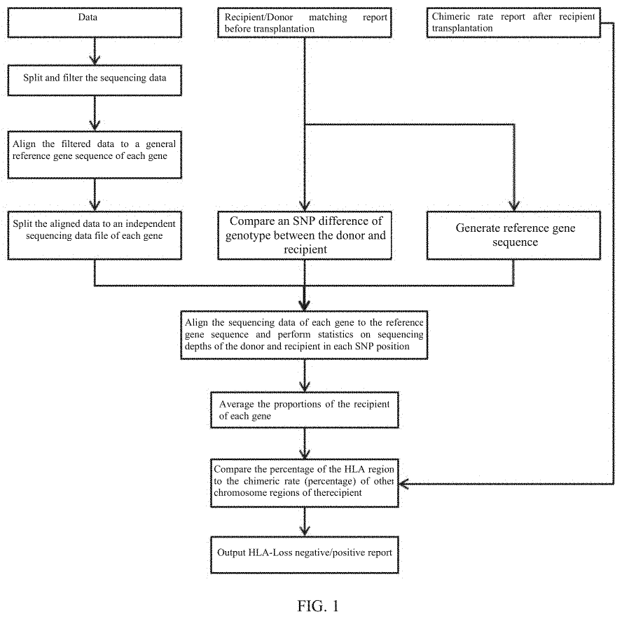

As shown in FIG. 1, FIG. 1 depicts an analysis method for HLA Loss provided by the present application, including the following steps:

splitting and filtering a sample data to obtain a filtered sample sequence;
acquiring a sequencing data file of each HLA gene, wherein the sequencing data file comprises the sample data of each HLA gene;
generating a first allele sequence as a reference gene before recipient transplantation;
acquiring a percentage occupied in each HLA gene after the recipient transplantation;
acquiring a percentage of the HLA gene chromosome region after the recipient transplantation, denoted by HLA %;
acquiring a total percentage of each chromosome in a cell after the recipient transplantation, denoted by STR %;
judging a negative or positive result of HLA Loss after the recipient transplantation.

As shown in FIG. 2. FIG. 2 depicts a display diagram showing a mutual relation of each module in an analysis processing apparatus for HLA Loss provided by the present application. The analysis processing apparatus includes the following modules:

a splitting and filtering module, used for splitting and filtering a sample data to obtain a filtered sample sequence;
a gene sequencing data file acquisition module, used for acquiring each HLA gene sequencing data file, wherein the sequencing data file comprises the sample data of each HLA gene;
a reference gene generation module, used for generating a first allele sequence of HLA genotype as a reference gene before recipient transplantation;
a calculation module for a percentage in each HLA gene, used for acquiring a percentage occupied in each HLA gene after the recipient transplantation;
a calculation module for a percentage of a HLA gene chromosome region, used for acquiring a percentage of a HLA gene chromosome region after the recipient transplantation, denoted by HLA %;
a calculation module for a total percentage of each chromosome of each HLA gene, used for acquiring a total percentage of each chromosome in a cell after the recipient transplantation, denoted by STR %;
a negative or positive judgment module, used for judging a negative or positive result of HLA Loss after the recipient transplantation.

The above analysis method and analysis processing apparatus for HLA Loss will be specifically described by the following examples.

### Example 1: Detection on a Negative HLA Loss Recipient after Hematopoietic Stem Cell Transplantation

1. Targeted NGS Detection on a Recipient after Hematopoietic Stem Cell Transplantation to Acquire Matching and Chimeric Results

A blood specimen from a recipient after hematopoietic stem cell transplantation was used in this example, and genome DNA was extracted as a template for the targeted NGS detection; the detection results were analyzed by the analysis method of the present application.

HLA matching results of the recipient before transplantation based on a pre-transplantation matching report were as follows: A*02:10, A*24:02, B*15:01, B*40:01, C*03:03, C*07:02, DRB1*09:01, DRB1*14:05, DQB1*05:03, DQB1*03:03; HLA matching results of a donor were as follows: A*24:02, A*26:01, B*15:01, B*40:01. C*03:03, C*07:02, DRB1*14:05, DRB1*15:02. DQB1*05:03, DQB1*06:01. B gene and C gene of the donor and recipient coincide with each other, and the method of the present application is only used to analyze and calculate genes A, DRB1 and DQB1. According to a chimeric rate report after transplantation, a ratio STR % of the recipient cells in blood was acquired 41.27%.

### 2. Splitting and Filtering the Sequence Obtained by Sequencing to Obtain a High-Quality Sample Sequence

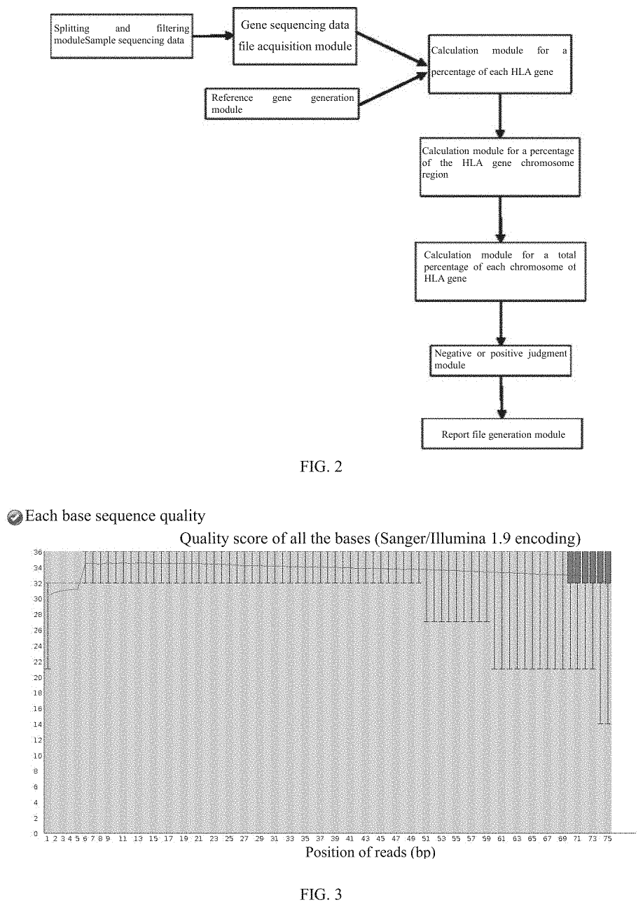

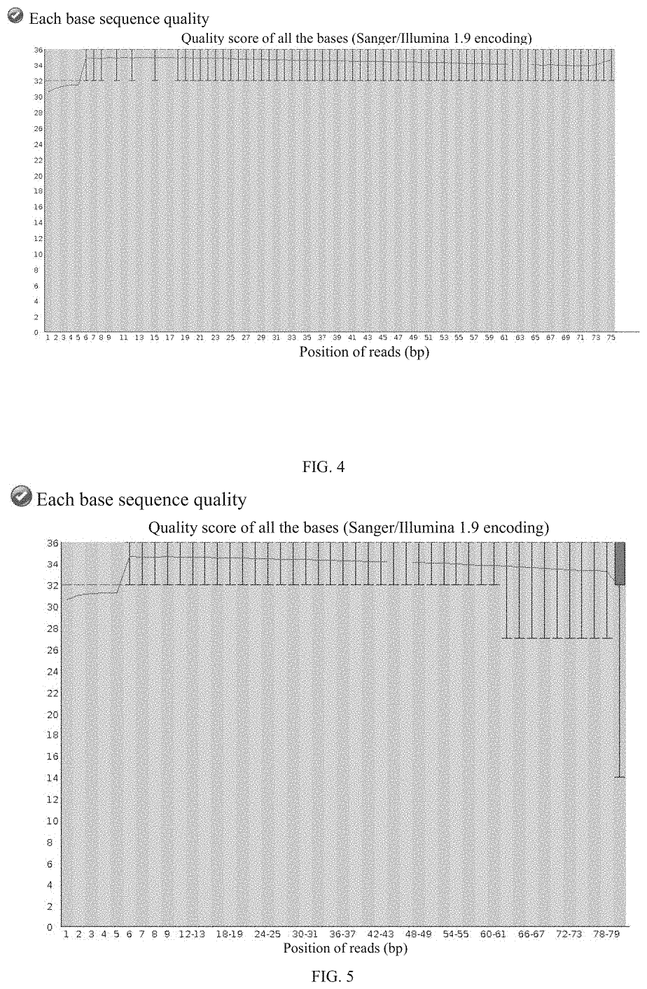

Referring to the flow diagram as shown in FIG. 1, bcl2fastq software (website: https://support.illumina.com/sequencing/sequencing_software/bcl2fastq-conversion-software.html) was used to split NGS downlink data, and Trimmomatic software (website: http://www.usadellab.org/cms/?page=trimmomatic) was used for filtering. Filtering parameters were as follows: a threshold of mean filter quality: 30; Sliding window: 5; the filtered sample sequence was obtained; the base number having a quality value lower than the threshold value was cut off on (Leading, Trailing): 5; a length threshold (Minlen): 50; and a high-quality sample sequence was obtained. The alignment before and after data filtering is shown in FIGS. 3-4. It can be apparently seen that the original data has partial reads on the bases 1 and 51-75 of the reference gene, and data quality declines, but after through data filtering, these poor-quality portions are all deleted.

### 3. Aligning the Filtered Sample Sequence to the Reference Gene of HLA Genes to Obtain a Sequencing Data File

Sequences A*01:01:01:01, DRB1*01:02:01:01 and DQB1*05:01:01:01 were obtained by querying IMGT-HLA common data base (website: https://www.ebi.ac.uk/ipd/imgt/hla/), and bwa software (website: https://sourceforge.net/projects/bio-bwa/) was used to align the filtered sample sequence in the above step to the reference gene of each HLA gene (default parameters were used for alignment), and the aligned sequencing sequence was split to 3 sequencing data files, namely. HLA-A, HLA-DRB1 and HLA-DQB1.

4. Comparison on an SNP Difference Between Different Types of Each Gene from the Recipient and Donor to Obtain snp Files

MUSCLE software (website: http://www.drive5.com/muscle/) was used to compare an SNP difference between 3 types of A*02:10, A*24:02 and A*26:01 in a gene A from the recipient and donor (default parameters were used for alignment), where there were 4 SNPs in total, saved as A.snp files; to compare an SNP difference between 3 types of DRB1*09:01, DRB1*14:05, and DRB1*15:02 in a gene DRB1 from the recipient and donor; where there were 37 SNPs in total, saved as DRB1.snp files; to compare an SNP difference between 3 types of DQB1*05:03. DQB1*03:03, DQB1*06:01 in a gene DQB1 from the recipient and donor; where there were 19 SNPs in total, saved as DQB1.snp files.

### 5. Aligning the Sequencing Data Files to the Reference Sequences of the Corresponding Genes Respectively to Generate Alignment Files of Each Gene

The reference sequences A*02:10, DRB1*09:01:02:01, DQB1*05:03:01:01 were obtained by querying the IMGT-HLA common data base (website: https://www.ebi.ac.uk/ipd/imgt/hla/), and the mem algorithm in the bwa software (website: https://sourceforge.net/projects/bio-bwa/) was used to respectively align the sequences in the 3 sequencing data files to the reference sequences A*02:10, DRB1*09:01:02:01, and DQB1*05:03:01:01 by genes, thus generating each gene comparison file A.bam, DRB1.bam and DQB1.bam.

### 6. Acquiring a Sequencing Depth in Each SNP Position of the Gene Alignment File to Calculate a Mean Value of Depth Proportions of Each Gene

Further, snp files were acquired and bamreadcount software (website: https://github.com/genome/bam-readcount) was used to acquire a sequencing depth in each SNP position and calculate a proportion of the depth belonging to the type of the recipient in each position. 4 SNPs in the gene A respectively account for 47.88%, 38.11%, 51.75% and 46.32% with a mean number of 46.02%; 18 SNPs have been detected in the 37 SNPs of the gene DRB1, and respectively account for 23.64%, 31.60%, 52.81%, 41.32%, 48.04%, 64.69%, 46.54%, 43.59%, 58.48%, 36.23%, 70.02%, 75.06%, 30.40%, 48.29%, 46.99%, 25.18%, 56.42% and 40.23% with a mean number of 46.64%; and 7 SNPs are detected in the 19 SNPs of the gene DQB1, and respectively account for 46.23%, 44.99%, 38.79%, 36.90%, 37.59%, 32.15% and 36.68% with a mean number of 39.05%.

7. Acquiring a Mean Value of all the Genes to Judge the Presence of HLA Loss or not in the Recipient after Transplantation

Further, 3 genes were averaged 43.90%. According to the negative or positive adjustment method of the present application, the proportion (43.90%) of the HLA Loss of the recipient after transplantation is greater than 3%, the detected is judged as negative HLA Loss, and the results show that the detected has no HLA Loss after transplantation.

### Example 2: Detection on a Positive HLA Loss Recipient after Hematopoietic Stem Cell Transplantation

1. Targeted NGS Detection on a Recipient after Hematopoietic Stem Cell Transplantation to Acquire

Matching and Chimeric Results A blood specimen from a recipient after hematopoietic stem cell transplantation was used in this example, and genome DNA was extracted as a template for the targeted NGS detection; the detection results were analyzed by the analysis method of the present application.

HLA matching results of the recipient before transplantation based on a pre-transplantation matching report were as follows: A*02:03, A*30:01, B*13:02, B*18:01, C*06:02, C*07:04, DRB1*07:01, DRB1*14:04, DQB1*05:03, DQB1*02:02; HLA matching results of a donor were as follows: A*11:01, A*30:01, B*13:02, B*15:02, C*06:02, C*08:01, DRB1*07:01, DRB1*13:02, DQB1*06:04, DQB1*02:02. According to a chimeric rate report after transplantation, a ratio STR % of the recipient cells in blood was acquired 6.85%.

### 2. Splitting and Filtering the Sequence Obtained by Sequencing to Obtain a High-Quality Sample Sequence

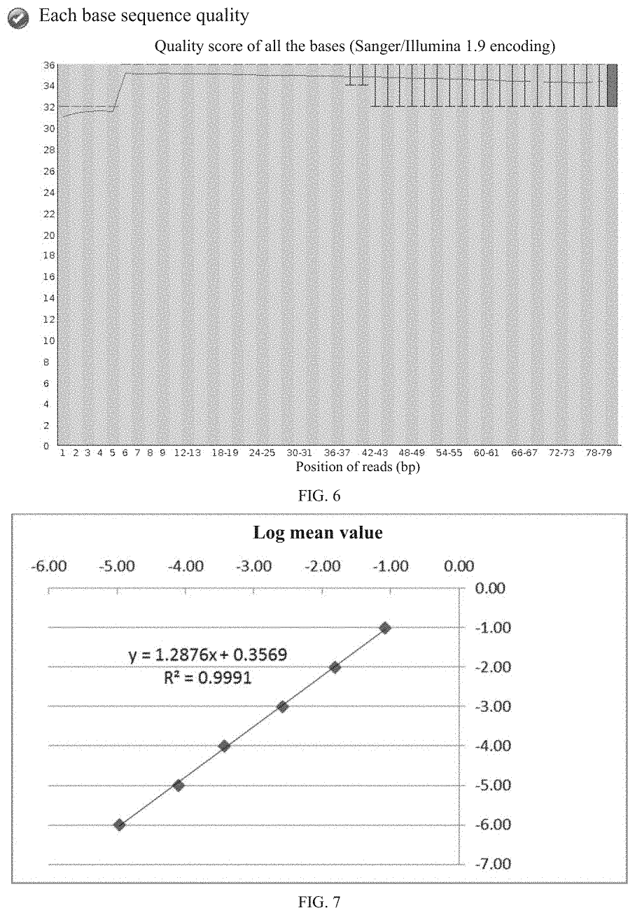

Referring to the flow diagram of FIG. 1, bel2fastq software (website: https://support.illumina.com/sequencing/sequencing_software/bcl2fastq-conversion-software.html) was used to split the NGS downlink data; Trimmomatic software (website: http://www.usadellab.org/cms/?page=trimmomatic) was used for filtering; and parameters were as follows: a threshold of mean filter quality: 30; Slidingwindow: 5; the filtered sample sequence was obtained; the base number having a quality value lower than the threshold value was cut off on (Leading, Trailing): 5; a length threshold (Minlen): 50; a high-quality sample sequence was obtained. The comparison before and after data filtering is shown in FIGS. 5-6. It can be apparently seen that the original data has partial reads on the bases 61-75 of the reference gene, and data quality declines, but after through data filtering, these poor-quality portions are all deleted.

### 3. Aligning the Filtered Sample Sequence to a Reference Gene of Each HLA Gene to Obtain a Sequencing Data File

Sequence A*01:01:01:01, B*07:02:01:01, C*01:02:01:01, DRB1*01:02:01:01, DQB1*05:01:01:01 was obtained by querying IMGT-HLA common data base (website: https://www.ebi.ac.uk/ipd/imgt/hla/), and bwa software (website: https://sourceforge.net/projects/bio-bwa/) was used to align the filtered sample sequence in the above step to the reference gene of each HLA gene (default parameters were used for alignment), and the aligned sequencing sequence was split to 5 sequencing data files, namely, HLA-A, HLA-B, HLA-C, HLA-DRB1 and HLA-DQB1.

4. Comparison on an SNP Difference Between Different Types in Each Gene from the Donor and Recipient to Obtain Snp Files

MUSCLE software (website: http://www.drive5.com/muscle/) was used to compare an SNP difference between 3 types of A*02:03, A*30:01 and A*11:01 in a gene A from the recipient and donor (default parameters were used for alignment), where there were 6 SNPs in total, saved as A.snp files; to compare an SNP difference between 3 types of B*13:02, B*18:01 and B*15:02 in a gene B from the recipient and donor; where there were 6 SNPs in total, saved as B.snp files; to compare an SNP difference between 3 types of C*06:02, C*07:04 and C*08:01 in a gene C from the recipient and donor; where there were 2 SNPs in total, saved as C.snp files; to compare an SNP difference between 3 types of DRB1*07:01. DRB1*14:04 and DRB1*13:02 in a gene DRB1 from the recipient and donor; where there were 9 SNPs in total, saved as DRB1.snp files; and to compare an SNP difference between 3 types of DQB1*05:03, DQB1*02:02 and DQB1*06:04 in a gene DQB1 from the recipient and donor; where there were 14 SNPs in total, saved as DQB1.snp files.

### 5. Aligning the Sequencing Data Files to the Reference Sequences of the Corresponding Genes Respectively to Generate Comparison Files of Each Gene

The reference sequences A*02:03:01, B*13:02:01:01, C*06:02:01:01, DRB1*07:01:01:01 and DQB1*05:03:01:01 were obtained by querying the IMGT-HLA common data base (website: https://www.ebi.ac.uk/ipd/imgt/hla/), and the mem algorithm in the bwa software (website: https://sourceforge.net/projects/bio-bwa/) was used to respectively compare the sequences in the 5 sequencing data files to the reference sequences A*02:03:01. B*13:02:01:01, C*06:02:01:01, DRB1*07:01:01:01 and DQB1*05:03:01:01 by genes, thus generating comparison files of each gene: A.bam, B.bam, C.bam, DRBL.bam and DQB1.bam.

### 6. Acquiring a Sequencing Depth in Each SNP Position of the Gene Alignment File to Calculate a Mean Value of Depth Proportions of Each Gene

Further, snp files were acquired and bamreadcount software (website: https://github.com/genome/bam-readcount) was used to view sequencing depths of each SNP position in the bam file and calculate a proportion of depths belonging to the type of the recipient in each position. 6 SNPs in the gene A respectively account for 0.53%, 0.52%, 0.35%, 0.35%, 0.52% and 0.38% with a mean number of 0.44%; 5 SNPs have been detected in the 6 SNPs of the gene B, and respectively account for 0.11%, 0.16%, 0.41%, 0.44% and 0.29% with a mean number of 0.28%; and 2 SNPs of the gene C respectively account for 0.21% and 0.45% with a mean number of 0.33%; 2 SNPs have been detected in the 9 SNPs of the gene DRB1, and respectively account for 0.00% and 0.43% with a mean number of 0.22%; 7 SNPs have been detected in the 14 SNPs of the gene DQB1, and respectively account for 0.37%, 0.13%, 0.14%, 0.24%, 0.14%, 0.08% and 0.24% with a mean number of 0.19%.

7. Acquiring a Mean Value of all the Genes to Judge the Presence of HLA Loss or not in the Recipient after Transplantation

Further, 5 genes were averaged 0.29%. According to the negative or positive adjustment method of the present application, the proportion (0.29%) of the HLA Loss of the recipient after transplantation is lower than 0.5% and the chimeric rate (6.85%) is greater than 3%, the recipient is judged as positive HLA Loss, and the results show that the recipient has HLA Loss after transplantation.

### Example 3: Verification on the Sensitivity and Specificity of the Algorithm of the Present Application

In this example, two samples from healthy subjects were artificially mixed in an equal proportion to simulate the chimeric rates of different donors and recipients; and the algorithm of the present application was used to detect the proportions of the sequencing results of the two samples at each concentration gradient, and linear fitting was used to verify the sensitivity and specificity of the algorithm of the present application.

In this example, two cases of blood specimens from healthy subjects were randomly selected; HLA matching results of sample one were A*26:01, A*31:01, B*40:06, B*40:06, C*08:01, C*15:02, DRB1*09:01, DRB1*15:02, DQB1*06:02 and DQB1*03:03; HLA matching results of sample two were A*24:02, A*24:02, B*40:01, B*40:03, C*03:04, C*07:02, DRB1*12:01, DRB1*15:01, DQB1*06:02 and DQB1*03:01; after the DNA concentration of samples was detected by a spectrophotometer, and based on the DNA concentration, the two samples were mixed into 6 different proportions of samples (1:1, 1:4, 1:8, 1:16, 1:32 and 1:64) by a concentration gradient dilution method, then next generation base building and sequencing were performed.

In the mixed samples, the sample one has theoretical proportions of 50.00%, 25.00%, 12.50%, 6.25%, 3.12% and 1.57% respectively. It should be indicated that the proportions are only reference values, and converted after detecting absorbance with the spectrophotometer and thus, are not actual proportions, only as mixed sample references, but not as valid reference values of the algorithm of the present application.

The analysis method of the present application is used for analyzing the downlink sequencing data of each proportion of sample; the proportion of each gene sample one is shown in Table 1; the mean value of the results is further subjected to linear fitting, and the results are shown in FIG. 7. The fitting result shows that the algorithm of the present application is used for the detection of HLA Loss to have rather high sensitivity and specificity.

TABLE 1
Table for gradient mixing results of Example 3

| Theoretical | Result | Result | Result | Result | Result | Mean |
|---|---|---|---|---|---|---|
| proportion | A | B | C | DRB1 | DQB1 | result |
| 50.00% | 49.49% | 49.01% | 45.95% | 46.34% | 45.78% | 47.31% |
| 25.00% | 29.67% | 28.77% | 25.77% | 77.59% | 30.27% | 28.41% |
| 12.50% | 18.14% | 19.41% | 13.88% | 14.92% | 17.54% | 16.78% |
| 6.25% | 11.42% | 10.04% | 7.62% | 7.03% | 10.14% | 9.25% |
| 3.12% | 8.74% | 5.43% | 4.65% | 3.20% | 7.15% | 5.83% |
| 1.57% | 5.07% | 2.93% | 2.30% | 1.48% | 4.23% | 3.20% |

1. An analysis method for HLA Loss, comprising the following steps:
(1) splitting and filtering a sample data to obtain a filtered sample sequence;
(2) acquiring sequencing data file of each HLA gene, wherein the sequencing data file comprises the sample data of each HLA gene;
(3) generating a first allele sequence as a reference gene before recipient transplantation;
(4) acquiring a percentage occupied in each HLA gene after the recipient transplantation;
(5) acquiring a percentage of the HLA gene chromosome region after the recipient transplantation, denoted by HLA %;
(6) acquiring a total percentage of each chromosome in a cell after the recipient transplantation, denoted by STR %; and
(7) judging a negative or positive result of HLA Loss after the recipient transplantation.

2. The analysis method according to claim 1, wherein each step is specifically as follows:
(1) splitting and filtering a sample downlink data after completing the sequencing to obtain the filtered sample sequence;
(2) based on a given alignment parameter, aligning the filtered sample sequence to a reference gene sequence of each HLA gene, and splitting the aligned sequencing sequence to each HLA sequencing data file; the sequencing data file comprises the sample downlink data of each HLA gene after completing the sequencing;
(3) acquiring the type of each HLA allele from a recipient and a donor before transplantation, after alignment, obtaining an SNP difference of each HLA gene between the recipient and the donor, and generating the first allele sequence of the HLA genotype before recipient transplantation as a reference gene;
(4) aligning the sequencing data file to the reference gene, performing statistics on respective sequencing depths of the recipient and the donor in a position SNP of each HLA gene, and averaging a depth frequency of the recipient in all the SNP positions of each HLA gene to obtain the percentage occupied in each HLA gene after the recipient transplantation;
(5) averaging the percentage of the recipient in each HLA gene, namely, a percentage of the HLA chromosome region after the recipient transplantation, denoted by HLA %;
(6) obtaining a total percentage of each chromosome in a cell after the recipient transplantation, denoted by STR %;
(7) negative or positive judgment: wherein, when HLA %50.5% and STR %≥3%, HLA Loss is judged positive; when HLA %≥3%, HLA Loss is judged negative; when 0.5%5 HLA %<3%, the failure of judgment is prompted, and the cell HLA chromosome may be in a deficiency phase after the recipient transplantation; and
(8) outputting the percentage and average percentage of each HLA gene, negative or positive judgment result to a report file.

3. The analysis method according to claim 1, wherein each HLA gene is respectively HLA-A, HLA-B, HLA-C, HLA-DRB1, HLA-DQB1 and HLA-DPB1.

4. The analysis method according to claim 2, wherein the HLA-A gene is aligned to A*01:01:01:01; the HLA-A gene is aligned to B*07:02:01:01; the HLA-C gene is aligned to C*01:02:01:01; the HLA-DRB1 gene is aligned to DRB1*01:02:01:01; the HLA-DQB1 gene is aligned to DQB1*05:01:01:01; and the HLA-DPB1 gene is aligned to DPB1*01:01:01:01.

5. An analysis processing apparatus for HLA Loss, comprising the following modules:
(1) a splitting and filtering module, used for splitting and filtering a sample data to obtain a filtered sample sequence;
(2) a gene sequencing data file acquisition module, used for acquiring the sequencing data file of each HLA gene, wherein the sequencing data file comprises the sample data of each HLA gene;
(3) a reference gene generation module, used for generating a first allele sequence as a reference gene before recipient transplantation;
(4) a calculation module for a percentage in each HLA gene, used for acquiring a percentage occupied in each HLA gene after the recipient transplantation;
(5) a calculation module for a percentage of a HLA gene chromosome region, used for acquiring a percentage of a HLA gene chromosome region after the recipient transplantation, denoted by HLA %;
(6) a calculation module for a total percentage of each chromosome of HLA gene, used for acquiring a total percentage of each chromosome in a cell after the recipient transplantation, denoted by STR %;
(7) a negative or positive judgment module, used for judging a negative or positive result of HLA Loss after the recipient transplantation.

6. The analysis processing apparatus according to claim 5, wherein each module is specifically as follows:
(1) a splitting and filtering module, used for splitting and filtering a sample downlink data after completing the sequencing to obtain the filtered sample sequence;
(2) a gene sequencing data file acquisition module, used for aligning the filtered sample sequence to a reference gene sequence of each HLA gene based on a given alignment parameter, and splitting the aligned sequencing sequence to each HLA sequencing data file; wherein the sequencing data file comprises the sample downlink data of each HLA gene after completing the sequencing;
(3) a reference gene generation module, used for acquiring a type of each HLA allele from a recipient and a donor before transplantation, after alignment, obtaining an SNP difference of each HLA gene between the recipient and the donor, and generating a first allele sequence of the HLA genotype as the reference gene before recipient transplantation;
(4) a calculation module for a percentage of each HLA gene, used for aligning the sequencing data file to the reference gene, performing statistics on respective sequencing depths of the recipient and the donor in a position SNP of each HLA gene, and averaging a depth frequency of the recipient in all the SNP positions of each HLA gene to obtain the percentage occupied in each HLA gene after the recipient transplantation;
(5) a calculation module for a percentage of the HLA gene chromosome region, used for averaging the percentage of the recipient in each HLA gene, namely, a percentage of the HLA chromosome region after the recipient transplantation, denoted by HLA %;
(6) a calculation module for a total percentage of each chromosome of each HLA gene, used for acquiring a total percentage of each chromosome in a cell after the recipient transplantation, denoted by STR %;
(7) a negative or positive judgment module: used for judging a negative or positive result of HLA Loss after recipient transplantation; when HLA %≤0.5% and STR %≥3%, HLA Loss is judged positive; when HLA %≥3%, HLA Loss is judged negative; when 0.5%≤HLA %<3%, the failure of judgment is prompted, and the cell HLA chromosome may be in a deficiency phase after the recipient transplantation; and
(8) a report file generation module, used for outputting the percentage and average percentage of each HLA gene, and negative or positive judgment result to a report file.

7. The analysis processing apparatus according to claim 6, wherein each HLA gene is respectively HLA-A, HLA-B, HLA-C, HLA-DRB1, HLA-DQB1 and HLA-DPB1.

8. The analysis processing apparatus according to claim 7, wherein the HLA-A gene is aligned to A*01:01:01:01; the HLA-A gene is aligned to B*07:02:01:01; the HLA-C gene is aligned to C*01:02:01:01; the HLA-DRB1 gene is aligned to DRB1*01:02:01:01; the HLA-DQB1 gene is aligned to DQB1*05:01:01:01; and the HLA-DPB1 gene is aligned to DPB1*01:01:01:01.
